(19) United States
(12) Patent Application Publication
(10) Pub. No.: US 2008/0167282 A1
(43) Pub. Date: Jul. 10, 2008

(54) Use of Inhibitors of Steroid Activity for the Treatment of Substance Related Disorders

(76) Inventor: Giuseppe La Pera, Rome (IT)

Correspondence Address: ARENT FOX LLP, 1050 CONNECTICUT AVENUE, N.W., SUITE 400, WASHINGTON, DC, 20036, US

(21) Appl. No.: 11/886,523

(22) PCT Filed: Mar. 17, 2006
(86) PCT No.: PCT/IT06/00168

§ 371 Date: Sep. 17, 2007

(30) Foreign Application Priority Data
Mar. 18, 2005 (IT) RM2005A000127

(51) Int. Cl. A61K 31/58; A61K 31/56; A61P 5/00

(52) U.S. Cl. 514/176; 514/178

(57) ABSTRACT

The invention concerns the use of one or more steroid activity inhibitors, namely androgens, such as, for example, testosterone, for the treatment of disorders related to, or resulting from use of, or induced by substances or induced by intoxication from substances or abstinence from substances, mental disorders induced by substances, substance addiction or gambling, abuse of substances or recurrent criminal behaviour.

The present invention concerns the use of inhibitors of the steroid activity for the treatment of substance related disorders. Particularly, the present Invention concerns the use of one or more steroid activity inhibitors, namely androgens, such as testosterone, for the treatment of disorders related to or resulting from use of or induced by substances or induced by intoxication from substances or abstinence from substances, mental disorders induced by substances, the substance addictions or gambling, abuse of substances or recurrent criminal behaviour.

It is well known that there are approximately 40 million drug addicts worldwide and such abuses cause severe impairment both at psychological and physical level, such as, for example, the loss of moral integrity, healthy damage, reduced lifetime and severe problems of abstinence once it is decided to stop. On the other hand, it is also a problem that interests the society and not only the single individual, since often those who misuse drug are more responsible for crimes in our society.

Consequently, drug abuse has severely threatened the progress and social security of the human society. Although various governments have attempted to contrast the phenomenon with measures prohibiting drug abuse, the problem is not only increasing but is also widespread.

It is well known that differences exist related to sex in the predisposition to drug addiction, such as cocaine. Hu M. et al. in a recent paper (Neuropsychopharmacology, 2003) showed that women are more predisposed to cocaine addiction than men. This has also been demonstrated in experiments on rats and it has been correlated with the different brain organization in the two sexes and the different effects of circulating sex hormones. For example, oestradiol would appear to increase dopamine release at striatum and nucleus accumbens level and contributes to the increase of cocaine vulnerability in female subjects.

In another paper by Caine S. B. et. al. (Neuropsychopharmacology, 2004), it has been demonstrated that sex and steroid gonadal hormones may influence the behaviour related to cocaine abuse in certain conditions, and the existence has been stressed of numerous examples of controversial data in the scientific literature. In fact, results show that sex, oestrogen and testosterone levels are not critical determinants in the enhancement of the effects of cocaine and therefore, are in contrast with results of previous studies. In particular, the study addresses the effects of possible modulation of testosterone on the enhancement of the cocaine effects (i.e hyperactivity), modulation that seems dependent upon the type of acute (Martinez-Sanchez et al. 2002) or chronic testosterone administration (Long et. al., 1994).

At present the main methods adopted for the treatment of the addiction in most countries are as follow:

### a) Reduction Regimen

The composition mainly in use is based upon opium. The dose of opium is gradually reduced to zero, in the various treatment steps, and administered to the patient.

### b) Replacement Therapy

In this case the treatment of choice is methadone which is used for the addicted patient, in particular heroin addicted.

For both addiction treatments severe side effects and disadvantages have emerged in the clinical practice. For example, in the first case a long treatment period it is necessary and moreover addiction is relieved but not completely eliminated and the percentage of relapse following the treatment is high. With to the second method, methadone can induce drug addiction, whereby 100 mg of methadone per os is equivalent to 10 mg of morphine administered i.v. . Moreover, methadone itself may trigger various side effects such pulmonary lesions or oedema, immunological symptoms, impotence, and in more severe cases even blindness in both eyes. Furthermore children born of methadone addicted mothers are also likely to be addicted. Therefore, the substitution therapy cannot be considered an ideal method in the treatment of addiction.

Currently various alternative drugs have been tested for the treatment of the drug addiction, such as for example: Amantidine Bupropione, Buspirone, Bromocriptine, Carbamazepine (Tegretol), Fluoxetine (Prozac), Flupentixol, Mazindolo Naltrexone. While some among them proved ineffective others are still under evaluation.

In the light of the above mentioned findings it is obvious new methods and/or effective drug are need for the control and treatment of addiction which do not lead to severe side effects and disadvantages of the products used so far.

Surprisingly the author of the present invention now disclosed that the use of steroid activity inhibitors, in particular androgens, such as for example testosterone, can be used advantageously for the treatment of the addictions, with particular reference to the heroin addictions, cocaine, smoking, amphetamines, alcohol, drugs, psychoanaleptics and central nervous system stimulating drugs, psycholeptics and drugs depressing the central nervous system or reducing the pain, psychodisleptics and drugs affecting perceptive functions and perception, ecstasy, LSD, fungi (peyote), substance abuse and gambling. Moreover, the author of the present invention disclosed that the above mentioned active principles can be used advantageously for the treatment of compulsive obsessive disorders, depressive disorders, aggressive behaviours and recurrent criminal habit.

The above mentioned facts result from empirical observations and analysis of a clinical experience carried out by the author of the present invention which successfully determined the correlation existing between the high circulating levels of testosterone and the predisposition to the drug addiction, mainly in male individuals.

In particular, the author of the present invention observed that the adolescence is one of the more hazardous periods for the beginning of substance abuse and drug addiction in young people. Numerous hypotheses have attempted to establish possible risk factors mainly based on the social, familiar distress and psychological problems. The author of the present invention observed that substance abuse and drug addiction can result from a sexual problem. This theory derives from a set of considerations: drug addiction occurs mostly during a period in which the puberty process is completed in young males, as it is known that in this life period changes in the personality and behaviour occur in parallel with testosterone production regulating the puberty; in epidemiological surveys young people interviewed answered in a significant percentage, that indeed a sexual disorder was one of the possible risk factors for substance abuse and late drug addiction (La Pera G. et. al., Journal of Sex and Marital therapy, March-April 2003, pp. 149-156); a different distribution exists between males and females in the drug addicts that would result from the fact that the main difference between men and women able to produce different behaviours is the presence of the testis in man and ovary in woman.

It is therefore an object of the present invention the use of one or more inhibitors of the production, biosynthesis or its regulation, transport, metabolism or bioavailability of steroids, in particular androgens, such as for example testosterone, for the preparation of a medicament for the treatment of disorders related to or resulting from use of or induced by substances or induced by intoxication from substances or abstinence from substances, mental disorders induced by substances, the substance addictions or gambling, abuse of substances or recurrent criminal behaviour. The above mentioned substances can be, for example, alcohol, amphetamines, caffeine, Cannabis, cocaine, hallucinogens, inhalants, nicotine, opiate, phencyclidine, sedatives, hypnotics, anxiolytics, drugs, psychoanalectics and drugs stimulating the central nervous system,. psycholeptics and drugs depressing the central nervous system or reducing the pain, psychodisleptics and drugs altering perceptive functions and perception, ecstasy, LSD, fungi (peyote).

The aforesaid inhibitors can act on the non genomic effects of steroids or on genomic effects. The inhibition of genomic effects can occur through inhibition of the synthesis and steroid cytoplasmic receptor, inhibition of the formation of the complex between steroid receptor and ligand, by means of receptor antagonists, inhibition of the activity of the complex between steroid receptor and ligand. Furthermore the inhibition of steroids activity can occur by means of inhibition of the genomic target the synthesis of which is regulated by the steroid-ligand complex.

One particular group of compounds which can be used according to the present invention are the inhibitors of testosterone production and activity.

The inhibition of testosterone production can occur by acting at hypophysis level using agonists of the LHRH synthesis or antagonists of the LHRH receptor, that is active principles able to operate directly on the feedback mechanism which is regulated mainly by the LHRH receptor at hypophysial level, or directly at gonadal level. Examples of compounds acting on the feedback mechanism at level of LHRH system are triptorelin, leuprorelin acetate, buserelin acetate, goserelin acetate. While inhibitors of testosterone production by means of action on the gonads are, as an example, ketoconazol, aminoglutamide.

The inhibitors of testosterone production are molecules able to markedly reduce the circulating levels of testosterone in order to obtain a chemical castration in male individuals until the same hormone levels are achieved, that can be obtained by of surgical castration (indicatively levels less than 1 ng/ml).

The inhibitors of testosterone activity are for example oestrogens, antiandrogens, such as bicalutamide, cyproterone acetate, flutamide, danazol.

The present invention concerns moreover the use of one or more inhibitors of the production, biosynthesis or its regulation, transport, metabolism or bioavailability of steroids as above described, in combination with other effective active principles for the treatment of addictions like, as an example, metadoxine, methadone hydrochloride, antidepressants.

An inhibitor example of steroid transport is mepartricine.

Among drugs preventing the transformation in a metabolite can be indicated all the drugs preventing the transformation of testosterone in dihydrotestosterone, in particular, as an exemplification, finasteride and dutasteride can be reported.

The inhibitors according to the present invention can be comprised in formulations such as, for example, tablets, capsules, solutions, suspensions, injectable compositions, suppositories, depot formulations, compositions for a controlled release of the active principle, as for example for one or three months. Inhibitors can be administered by any suitable mode, as for example, oral, intravenous, intramuscular, rectal, transdermal, transurethral, intraurethral, nasal route.

The present invention now will be described according to preferred but not limiting embodiments thereof.

### EXAMPLE 1

### Study on the Smoking Addiction

In the clinical experience as urologist, the author of the present invention, has observed that in patients suffering from prostate cancer (100 patients) and subjected to total androgen blockade (50 patients), the percentage of subjects who stopped smoking with respect to those not receiving the treatment of total androgen blockade was meaningfully more elevated with a rate, respectively, of 56% in first vs 8% in the second ones.

### ESEMPIO 2

### Study on the Cocaine Addiction

25 year old, overweight, hirsutic female with polycystic ovary. In anamnesis the patient used cocaine several times over a week. The patient did not suffer from diabetes neither other important disease.

Due to the hirsutism, the patient was treated with cyproterone acetate 50 mg 1 cp daily. In the course of the follow up transaminases and hormones were assayed. The treatment lasted approximately 9 months. After 6 months of therapy the girl was “drug free” and no longer used cocaine.

### BIBLIOGRAPHY

Caine S B, Bowen C. to, Yu G., Zuzga D., Negus S S, Mello N K. Neuropsychopharmacology 2004; 29:92-942.
Hu M., Crombag H S, Robinson Y O U, Becker J B Neuropsychopharmacology 2003.
Martinez-Sanchez To, argon C M, Salvador To. Physiol. Behav. 2002; 76:605-609.
Long S F, Dennis T A, Russell R K, Wilson M C. Behav. Pharmacol. 1994; 5:103-106.

1. Use of one o more inhibitors of the production, biosynthesis or regulation thereof, transport, metabolism or bioavailability of steroids for the preparation of a medicament for the treatment of disorders related to or resulting from use of or induced by substances or induced by intoxication from substances or abstinence from substances, mental disorders induced by substances, the substance addictions or gambling, abuse of substances or recurrent criminal behaviour.

2. Use according to claim 1, wherein steroids are androgens.

3. Use according to claim 1, wherein androgen is testosterone.

4. Use according to claim 1, wherein the substances are selected form the group consisting of alcohol, amphetamines, caffeine, Cannabis, cocaine, hallucinogens, inhalants, nicotine, opiate, phencyclidine, sedatives, hypnotics, anxiolytics, drugs, psychoanalectics and drugs stimulating the central nervous system, psycholeptics and drugs depressing the central nervous system or reducing the pain, psychodisleptics and drugs altering perceptive functions and perception, ecstasy, LSD, fungi (peyote).

5. Use according to claim 1, wherein one or more inhibitors are associated with other effective active principles for the treatment of substance addictions or abuse.

6. Use according to claim 5, wherein other effective active principles for the treatment of substance addictions or abuse are metadoxine, methadone hydrochloride, antidepressants.

7. Use according to claim 1 wherein inhibitors act on non genomic effects of steroids or on genomic effects.

8. Use according to claim 7, wherein inhibitors of the genomic effects are inhibitors of the synthesis of steroid cytoplasmic receptor, inhibitors of the formation of the complex between steroid receptor and ligand, inhibitors of the activity of the complex between steroid receptor and ligand, inhibitors of genomic target whose synthesis is regulated by steroid receptor-ligand complex.

9. Use according to claim 1, wherein inhibitors are inhibitors of testosterone production and activity.

10. Use according to claim 9, wherein inhibitors of testosterone production are selected from the group consisting of triptorelin, leuprorelin acetate, buserelin acetate, goserelin acetate, ketoconazole, aminoglutamide.

11. Use according to claim 9, wherein inhibitors of testosterone activity are selected from the group consisting of oestrogens, antiandrogens.

12. Use according to claim 11, wherein antiandrogens are selected from the group consisting of bicalutamide, cyproterone acetate, flutamide, danazol.

13. Use according to claim 1, wherein inhibitor of steroid transport is mepartricin.

14. Use according to claim 1, wherein the drugs that prevent transformation in a metabolite are all the drugs preventing the transformation of testosterone in dihydrotestosterone, in particular, finasteride and dutasteride.

15. Use according to claim 1, wherein inhibitors are comprised in formulations selected from the group consisting of tablets, capsules, solutions, suspensions, injectable compositions, suppositories, depot formulations, controlled release compositions.

16. Use according to claim 15, wherein the controlled release compositions are lasting for one or three months.

17. Use according to claim 1 wherein inhibitors are administered by oral, intravenous, intramuscular, rectal, transdermal, transurethral, intraurethral, nasal route.
